(19) United States
(12) Patent Application Publication
(10) Pub. No.: US 2004/0111058 A1
(43) Pub. Date: Jun. 10, 2004

(54) Airless vent valve

(76) Inventors: Kenneth A. Jones, McKinney, TX (US); Darin John Miller, Rowlett, TX (US)

Correspondence Address: CARSTENS YEE & CAHOON, LLP, P O BOX 802334, DALLAS, TX, 75380

(21) Appl. No.: 10/726,400

(22) Filed: Dec. 3, 2003

Related U.S. Application Data
(60) Provisional application No. 60/430,390, filed on Dec. 3, 2002.

(51) Int. Cl. A61M 1/00

(52) U.S. Cl. 604/122; 604/35

(57) ABSTRACT

A system and method for controlling fluid flow in an extracorporeal circuit. A valve prevents positive pressure and negative pressure in a vent line by allowing entry of fluid when negative pressure occurs, and allowing exit of fluid from the line when positive pressure occurs. The valve preferably does this without introducing air into the circuit. The innovative valve also reduces homolysis by reducing turbulence in fluids passing through the valve.

### CROSS-REFERENCE TO RELATED APPLICATION

This application claims the benefit of and priority to a U.S. Provisional Patent Application No. 60/430,390 filed Dec. 3, 2002, the technical disclosure of which is hereby incorporated herein by reference.

### BACKGROUND OF THE INVENTION

1. Technical Field

The present invention relates to pump systems for open heart surgery, and particularly to valves used in such surgery.

2. Description of Related Art

In the performance of open heart surgery, the heart is generally bypassed and the patient's blood circulation is maintained by the use of a heart-lung machine such as a cardioplegia pump or similar apparatus. During such an operation, blood often finds its way into the left ventricle of the heart. This blood must be removed to avoid distending the left ventricle, which can make resuscitation of the heart at the end of the surgery difficult or impossible.

Drainage of the left ventricle is achieved by inserting a drainage cannula into the left ventricle. This cannula is used to drain blood either by gravity or by use of a pump or a combination of the two. The blood is typically directed through one or more conduits to a cardiotomy reservoir, and then to an oxygenator which oxygenates the blood. The blood flow is then directed back to the patient.

The rate at which a left ventricle pump operates determines the rate at which fluid is removed from the left ventricle. If the rate of pumping is to slow, fluid will accumulate in the left ventricle despite the pump, while if the rate of pumping is too great, the mouth of the drain tube can suck against the tissue of the heart, causing trauma.

One typical way to deal with this issue is to a positive-displacement aspiration pump and to control its operational speed throughout the surgical procedure. To control the level of suction applied, a negative pressure relief valve is used in the suction line between the pump and the heart. Such a valve typically controls the negative pressure by venting air into the suction line downstream of the valve if the suction line becomes occluded or otherwise develops too great a negative pressure. Negative pressure relief valves often include a check valve safety feature which prevents reverse flow of clued toward the heart.

Venting air into the suction line is not desirable, as air introduced into the blood flow system can introduce particulates or other pollutants, and can also increase chances of coagulation in the system. Such valves must also be monitored visually in order to determine if an occlusion in the vent line has occurred so that the occlusion can be dealt with and venting of the left ventricle restored without damage to the heart.

The art would therefore benefit from a vent valve that allows relief of both positive and negative pressure and which does not entrain air into the extracorporeal circuit.

### SUMMARY OF THE INVENTION

The present invention provides a system for venting excess fluid from a patient during heart surgery. In a preferred embodiment, the innovative system includes a valve between a pump and the patient. The preferred valve allows reduction of negative pressure in the line by allowing entry of fluid from a reservoir into that line when excess negative pressure occurs. In preferred embodiments, the valve has two inlets and one outlet. A first inlet accepts fluid from the patient, and a second inlet accepts fluid from a reservoir only when negative pressure exceeds a predetermined amount. An outlet allows fluid to pass from the patient to the pump, such as a vent pump.

Another innovative aspect of the present invention is the reduction of homolysis by providing fluid access at angles which reduce turbulence within the valve. By promoting smooth fluid passage through the valve, blood cells undergo less stress and are more likely to pass through the valve undamaged.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of an illustrative embodiment when read in conjunction with the accompanying drawings, wherein:

FIG. 1 shows an extracorporeal circuit consistent with a preferred embodiment.

FIG. 2 shows a detail of the innovative valve according to a preferred embodiment.

FIG. 3 shows the innovative valve in the context of the vent line and venous reservoir line.

FIG. 4 shows a detail of the innovative valve according to a preferred embodiment.

### DETAILED DESCRIPTION OF THE INVENTION

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

The present innovations are described with reference to the figures.

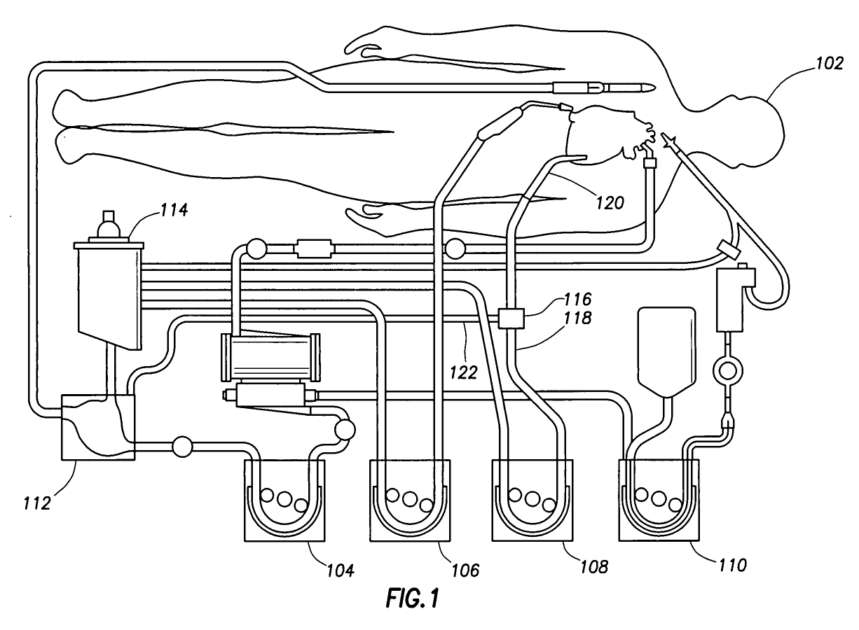

FIG. 1 shows a patient 102 with apparatus for performing heart surgery where the blood is circulated in an extracorporeal circuit. Four pumps are shown, an arterial pump 104, a sucker pump 106 for manual suction, a vent pump 108 which typically removes excess fluid from the left ventricle of the heart, and a cardioplegia pump 110 which provides cardioplegia solution and potentially other fluids to the patient. Also shown in the diagram are reservoirs, among them the venous reservoir 112 and the cardiotomy reservoir 114.

The present inventive concept is preferably employed as a valve 116 between vent pump 108 and the patient 102. The vent pump 108 connects to the patient via a line 118 which ends in a vent cannula 120 inserted into the left ventricle of the patient. The vent pump 108 is typically used to drain excess fluid from the left ventricle during surgery. Some means must be employed to prevent retroflow of fluid back into the patient, and to prevent excess vacuum from developing in the vent line 118 in case the vent cannula 120 or the line 118 itself becomes occluded. Valve 116 is therefore located in the vent line 118.

In a preferred embodiment, valve 116 is an airless vent valve which not only prevents retroflow back toward the patient and excess vacuum in the line 118, but it also preferably prevents excess positive pressure from building in the line 118 as well. In a preferred embodiment, the valve 116 achieves this goal without introducing air into the circuit. The valve 116 therefore has a line 122 which provides fluid access to the venous reservoir 112.

In its preferred operation, valve 116 allows fluid flow from patient 102 to the vent pump 108 but prevents flow in the opposite direction. Valve 116 also allows flow of fluid from venous reservoir 112 toward vent pump 108 in the event that excess negative pressure builds up in line 118. This function is referred to as negative pressure relief or excess vacuum relief of line 118. Valve 116 also provides positive pressure relief from the line 118 in the event of a buildup of positive pressure. In this event, valve 116 allows flow of fluid from the vent pump 108 toward the venous reservoir 112 (or another appropriate reservoir, depending on the implementation used, such as cardiotomy reservoir 114).

Inovative valve 116 therefore preferably provides negative pressure relief, positive pressure relief, and retroflow protection. It does this without introducing air into the circuit by allowing fluid to flow into the line 118 when the pressure exceeds a predetermined amount. Valve 116 also preferably offers a visual cue to when it performs these functions. Examples of possible visual cues include but are not limited to: transparency in the valve so that fluid flow can be detected; an electronic detector which monitors flow through the valve and provides an indication such as a display or light; or a mechanical indicator such as a button which pops out when the valve is activated for these functions, for example.

Valve 116 can provide positive pressure relief in other ways, such as by expelling fluid from the valve 116 to open air. this embodiment alleviates the need for a bi-directional flow at one inlet of the valve 116.

Though FIG. 1 shows the valve 116 connected to venous reservoir 112, alternative embodiments supply fluid to the valve via line 122 from an alternative source. This alternative source is preferably part of the closed extracorporeal circuit and is not open to atmosphere. Less preferred embodiments use the innovative valve 116 with reservoirs that are open to the atmosphere, but are still within the innovative concepts herein disclosed.

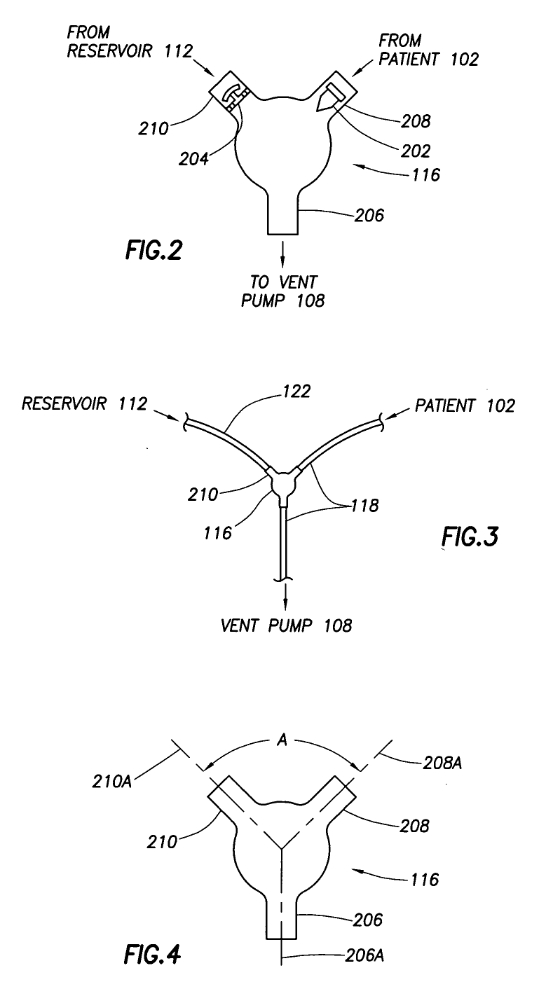

FIG. 2 shows a detail of the valve 116. Valve 116 is shown with three openings, a first inlet 210, a second inlet 208, and an outlet 206. Inlet 210 preferably attaches to a line that connects to reservoir 112 or similar reservoir. Inlet 208 preferably connects to patient 102 via line 118. Outlet 206 preferably connects to vent pump 108.

Inlet 208 preferably allows fluid flow only from patient 102 and prevents retroflow toward patient 102. This is achieved in a preferred embodiment with a one way valve 202 such as a duck billed valve as shown, for example.

If the vent cannula 120 becomes attaches to the tissue of the heart, trauma can result from the suction against tissue. This situation will create negative pressure inside line 118. Valve 116 relieves such negative pressure by admitting fluid into line 118 via inlet 210. Inlet 210 includes a valve 204 that automatically allows fluid flow from reservoir 112 toward vent pump 108 once negative pressure reaches a certain amount. The pressure required to allow this fluid flow is preferably determined by the mechanical structure of valve 116.

In an alternative embodiment, inlet 210 possesses only a one-way valve and is capable of relieving negative pressure in the line by admitting fluid from source 112. In this embodiment, valve 204 is preferably implemented as an umbrella valve or other one-way valve that responds to negative pressure on the side of the valve closest to line 118. When negative pressure in line 118 reaches a predetermined limit, valve 204 responds by admitting fluid into line 118 from source such as venous source 112. There is preferably a visual indicator function or other means in valve 116 to alert a monitor (human or otherwise) that negative pressure has occurred in the line and that the relief function has begun. For example, valve 116 could have a transparent window near valve 204 which will show when fluid passes through valve 204. Other indicators can be implemented as well, including electronic monitors and auditory alerts, depending in the implementation.

Valve 204 can also be variable in its response to negative pressure. Instead of a fixed pressure at which valve 204 admits fluid, some embodiments include a variable mechanism to controlling the negative pressure at which valve 204 activates.

FIG. 3 shows a simple diagram of the innovative valve system and nearby lines. Valve 116 is located on line 118 between patient 102 and vent pump 108. If vent cannula 120 becomes occluded, negative pressure builds up in line 118 by action of vent pump 108. In this instance, the negative pressure in line 118 will cause a valve at inlet 210 to activate, allowing flow from line 122 into line 118 thereby relieving the negative pressure and preventing trauma to heart tissue. Though line 122 is shown linking to venous reservoir 112, other reservoirs can be used. The reservoir used is preferably part of the extracorporeal circuit and is preferably closed to air.

FIG. 4 shows another innovative aspect of valve 116. This diagram shows inlets 208 and 210 and outlet 206 with their respective axes 208A, 210A, and 206A. The axes are represented as being parallel with the direction of fluid flow through each of the orifices. In order to reduce homolysis, the axes 210A and 208A enter valve 116 at an angle of less than ninety degrees with respect to one another. Supplying blood flow at angles so that the blood must change direction as little as possible reduces turbulence within the valve and thereby reduces damage to the cells within the blood.

The innovative valve system provides several advantages over prior valve systems, including alleviating the need to entrain air into the extracorporeal circuit, which reduces protein denaturation and activation of coagulation factors. It also reduces homolysis by virtue of the angle at which different flows pass through the valve. By relieving both positive and negative pressure in the line from the same source, no blood is lost from the extracorporeal circuit.

We claim:

1. A system of pumps, lines, and valves that circulate fluid outside a patient's body during a surgery, comprising:
a pump which draws fluid from the patient using suction;
a valve disposed in a first line connecting the pump and the patient, the first line having a first end being disposed inside the patient and a second end connected to the pump;
wherein when the first line experiences a predetermined amount of negative pressure, the valve allows fluid to flow from a source other than the patient toward the pump without introducing air into the first line.

2. The system of claim 1, wherein the valve allows fluid to flow from a source other than the patient toward the pump via a second line connecting to the first line at the valve.

3. The system of claim 1, wherein the valve presents a visual indicator when it allows fluid to flow from a source other than the patient into the first line.

4. The system of claim 1, wherein the valve relieves positive pressure in the first line.

5. The system of claim 1, wherein the valve prevents flow from the pump to the patient.

6. The system of claim 1, wherein the predetermined level is adjustable.

7. A valve for controlling pressure of fluid in a line of an extracorporeal circuit during heart surgery, comprising:
a first inlet having an axis parallel to the direction of fluid flow through the first inlet;
a second inlet having an axis parallel to the direction of fluid flow through the second inlet;
an outlet having an axis parallel to the direction of fluid flow through the outlet;
wherein the first inlet allows fluid suctioned from a patient's body to pass into the valve toward the outlet, but does not allow fluid flow in the reverse direction;
wherein the second inlet allows fluid to pass from a source into the valve toward the outlet in the event that negative pressure in the line reaches a predetermined level; and
wherein the axis of the first inlet and the axis of the second inlet have an angle between them less than ninety degrees.

8. The valve of claim 7, wherein the second inlet relieves negative and positive pressure in the line without introducing air into the line.

9. The valve of claim 7, wherein the valve presents a visual indicator when it allows fluid to flow from a source other than the patient into the first line.

10. The valve of claim 7, wherein the second inlet allows fluid to pass from the line to the source in the event that positive pressure in the line reaches a predetermined level.

11. The valve of claim 10, wherein the predetermined level is adjustable.

12. An extracorporeal circuit, comprising:
a pump in fluid communication with a first line, the first line being positioned to draw fluid from a patient;
a valve system in the first line between the pump and the patient, the valve system having a first valve which prevents flow of fluid from the pump toward the patient and a second valve which allows fluid to pass through the second valve toward the pump when negative pressure in the first line exceeds a predetermined amount;
wherein the valve system provides for relief of excess positive pressure in the first line; and
wherein the valve system relieves negative pressure by allowing fluid flow from the first source to the first line.

13. The circuit of claim 12, wherein the valve system relieves negative pressure in the first line without introducing air into the circuit.

14. The circuit of claim 12, wherein the valve system presents a visual indicator when it allows fluid to flow from a source other than the patient into the first line.

15. The circuit or claim 12, wherein the valve system relieves positive pressure by allowing fluid flow from the first line toward a first source.

16. The circuit of claim 12, wherein the predetermined amount is adjustable.

17. The circuit of claim 12, wherein no valve in the circuit introduces air into the circuit.
